(19) United States
(12) Patent Application Publication
(10) Pub. No.: US 2004/0161361 A1
(43) Pub. Date: Aug. 19, 2004

(54) Apparatus and method for sterilization of medical equipments, pharmaceutical products and biologically contaminated articles

(76) Inventors: Han Sup Uhm, Potomac, MD (US); Jung G. Kang, Suwon (KR)

Correspondence Address: Han Sup Uhm, 11613 Swains Lock Terrace, Potomac, MD, 20854, US

(21) Appl. No.: 10/365,404

(22) Filed: Feb. 13, 2003

(51) Int. Cl. A61L 2/20

(52) U.S. Cl. 422/30; 422/186.12; 422/292

(57) ABSTRACT

The invention is directed to an apparatus made of atmospheric resistive-barrier discharge for sterilization of medical tools wrapped in typical hospital cloths, for sterilization of manufactured drugs in typical packaging materials and for sterilization of biologically contaminated articles. The apparatus consists of layers of the resistive-barrier discharge device operated at room temperature. An electrical discharge in the resistive-barrier discharge system generates atmospheric plasma in oxygen gas, efficiently generating ozone, which in turn sterilizes the medical tools, manufactured drugs and biologically contaminated articles at room temperature.

### FIELD OF THE INVENTION

The present invention relates generally to the efficient ozone generation at room temperature and, in particular, to an apparatus and process for sterilization of the medical tools, manufactured drugs and biologically contaminated articles. Germs, viruses and pathogenic bacteria are killed by their exposure to the ozone in the oxygen plasma generated by the resistive-barrier discharge at room temperature and at the atmospheric pressure

### BACKGROUND OF THE INVENTION

Ethylene oxide (EO) is commonly used to sterilize the medical tools, which are sensitive to heat and water. Therefore, EO is very popular for sterilization of the medical tools at low temperature. However, in general, EO irritates eyes, causes the respiratory ailments, headache, diarrhea and vomit if inhaled, and also causes skin irritations by contact. It also leads to functional disorder of the central nerve system if inhaled. In addition, the ethylene oxide forms N-7 hydroxyethylguanine by a covalent bond with N-7 position DNA of guanosine. This N-7 hydroxyethylguanine occasionally causes transmutations, developing a cancerous cell. The N-7 hydroxyethylguanine also forms N-3-(2-hydroxyethyl)-histidine by a chemical combination with histidine in the hemoglobin. Thus, The American Conference of Governmental Industrial Hygienists (ACGIH) categorizes EO as an A2 (Suspected Carcinogen) material. In this context, it is necessary to find a new method for sterilization of medical tools and manufactured drugs at a low temperature instead of the ethylene oxide, a suspected carcinogen.

As a new way of low temperature sterilization, the U.S. Pat. No. 5,084,239, issued to Moulton et al. on Jan. 28, 1992, proposed the plasma sterilization in a low pressure chamber where the gaseous antimicrobial agent and plasma sterilize an article. Another sterilization method of medical instruments was proposed in the U.S. Pat. No. 5,266,275 issued to Faddis on Nov. 30, 1993, where ozone was introduced into the primary sterilization chamber. According to the U.S. Pat. No. 6,096,266 issued to Duroselle on Aug. 1, 2000, hydrogen peroxide with ozone is also very effective for sterilization. The U.S. Patent with U.S. Pat. No. 6,387,241 issued to Murphy, et al on May 14, 2002 also introduced method of sterilization using ozone. Obviously, ozone is very effective for killing germs, viruses and pathogenic bacteria. It is recommended to generate ozone continuously during sterilization. Dry sterilization of medical devices was proposed by the U.S. Pat. No. 6,149,878, issued to Jacob, et al on Nov. 21, 2000, where ozone was generated in reduced pressure by the electrical discharge in microwave cavity or by the electrode excitation by RF frequency, which requires complicated high-power equipments. The U.S. Pat. No. 6,007,770, issued to Peiper, et al. on Dec. 28, 1999, was sterilization of objects in a closed vessel placed between two high-voltage electrodes, which limit vessel size and therefore the sterilization volume for practical value of high-voltage. Sterilization by interposing a sterilization object between the electrodes and by causing a pulse streamer discharge was proposed by the U.S. Pat. No. 6,497,839 issued to Hasegawa, et al. on Dec. 24, 2002, however this invention also limits the sterilization volume.

It is therefore required to develop a practical ozone generation method for sterilization at room temperature and at the atmospheric pressure. Ozone is commonly made by an electrical discharge in an oxygen environment. There are conventional ozone generators commercially available, which are expensive and bulky. The dielectric-barrier discharge (DBD) is one kind of the corona discharge and is a new way to produce ozone. The dielectric-barrier discharge is an alternating current (ac) discharge with a dielectric material inserted in space between the two electrodes. This dielectric material prevents arcing and sustains an alternating discharge inside the electrodes. The plasma generated by a discharge with a good dielectric material is not uniform and not reliable, because the dielectric surface and the electrodes form capacitances, which are new elements of oscillation. Instead, this invention uses non-perfect dielectric medium (a resistive medium). A resistive medium is inserted into the space between the two electrodes. Not only the resistive medium prevents the arcing but also damps out any unstable oscillatory behavior in the discharge, stabilizing the discharge pattern and creating uniform stable plasma. The resistive-barrier discharge can be operated by either de or ac mode. The advantages of the present invention are the electrical power source and large area of the discharge surface, where a meshed wire electrode made of non-oxidizing and electrically conducting material creates a large volume of uniform plasma. This invention uses an inexpensive commercially available transformer with 15 kV, 20 mA and 60 Hz, which provides a dc electrical source by rectifying or an ac electrical source, depending on the applications. A large area of meshed-wire electrode creates a uniform, large area discharge, eliminating the edge effects and producing a stable discharge space. Most of the electrical power is converted into heat during an electrical discharge, raising the gas temperature in the vicinity of discharge area and also raising the barrier temperature at an electrode. However, this invention uses a large meshed-wire electrode spreading the discharge area, thereby dissipating generated heat very efficiently and holding down the gas temperature to room temperature, even without any gas flow for purpose of cooling. The given space can be used efficiently by stacking layers of the resistive-barrier discharge unit

It is therefore an important object of the present invention to create a uniform and large-area plasma in a resistive-barrier discharge in order to achieve sterilization of medical tools, manufactured drugs and biologically contaminated articles by exposure to ozone generated by electrical discharge in oxygen gas.

Other object of the present invention is to generate ozone at room temperature in an isolated chamber for a sterilization apparatus that is effective against a wide range of medical tools and manufactured drugs.

Another object of the present invention is to spread out the discharge area in the discharge device in order to efficiently dissipate the heat generated from the electrical discharge, thereby keeping the gas temperature in the sterilization chamber at room temperature.

Additional objects, advantages and novel features of the invention will be explained in part in the following description, and will be apparent to those skilled in the following experiments.

### SUMMARY OF THE INVENTION

The present invention is the apparatus for sterilization of medical tools wrapped in typical hospital cloths, manufactured drugs in typical packaging materials and biologically contaminated articles. Particularly, the apparatus is useful for sterilization of the medical tools, manufactured drugs and biologically contaminated articles at room temperature. The sterilization apparatus in this invention can be very useful for a large volume of manufactured drugs.

The present invention is making use of the corona discharge through a resistive medium inserted into the space between two electrodes operated by either de or ac electrical source. Ozone abundantly generated from the corona discharge in oxygen gas sterilizes the medical tools, manufactured drugs and biologically contaminated articles at a low temperature less than 35 degree Celsius. Particularly, the present invention will replace the conventional low-temperature sterilization by ethylene oxide gas, Sterilization verification of the present invention has been carried out by the ampule called Verify® Self-Contained Biological Indicators (SCBls), which are designed to monitor steam or any other sterilization processes. Each Verify Indicator consists of a plastic cap and vial containing a disc carrying Bacillus stearothermophilus (BST) and Bacillus subtilis (BSN) spores, and an ampule of bacterial culture medium combined with a pH indicator. In addition, these biological indicators are available in single species E6 populations. A color change from blue to yellow after activation and incubation gives unmistakable evidence of microbial growth. Sterilization verification of the present invention is carried out by Verify® SCBIs with 24 hour incubation time. Sterilization of the present invention is positively confirmed for verifying ampule stayed longer than 5 hours in the sterilization chamber of the invention.

One advantage of the present invention is ozone generation at a low temperature for sterilization, which is carried out by a large area of meshed-wire electrode, spreading out the discharge area and efficiently dissipating heat generated from the electrical discharge. The other advantage of the present invention is uniform plasma without any edge effects due to a large area of discharge space. The given space can be efficiently utilized by stacking layers of the resistive-barrier discharge unit.

### BRIEF DESCRIPTION OF DRAWING FIGURES

A more complete appreciation of the invention and many of its attendant advantages will be aided by reference to the following detailed description in connection with the accompanying drawings:

FIG. 1 is a block diagram illustrating the sterilization system of the medical tools, manufactured drugs, and biologically contaminated articles of the present invention.

FIG. 2 is a cross sectional view of the resistive-barrier discharge which generates ozone in oxygen gas.

FIG. 3 is a schematic presentation of the sterilization apparatus of the present invention.

FIG. 4 is a schematic presentation of the ozone destruction device of the present invention.

FIG. 5 is an example of the gas temperature versus time in the sterilization chamber of the present invention.

### DETAILED DESCRIPTION OF PREFERRED EMBODIMENTS

The present invention is the apparatus and method for sterilization of the medical tools wrapped in typical hospital cloths, manufactured drugs in typical packaging materials and biologically contaminated articles. The principles and operation of the sterilization apparatus of the present invention are described according to the drawings.

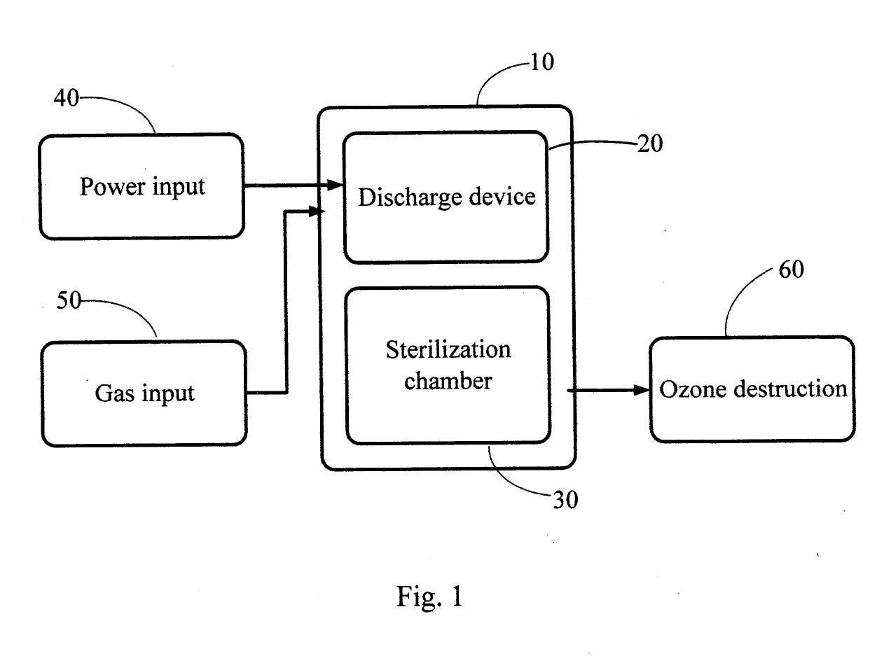

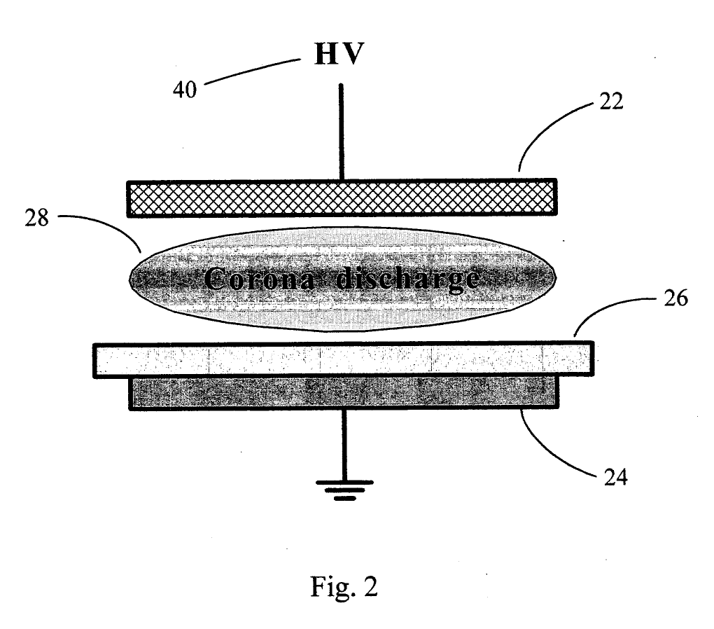

Referring now to the drawing in details, FIG. 1 diagrams the present invention wherein the oxygen gas 50 enters the discharge device 20 made of layers of the resistive-barrier discharge unit. The power input 40 provides the electrical power to the discharge device 20 for corona discharge. The sterilization chamber 30 is usually located under the discharge device 20. Note that ozone molecules are heavier than the oxygen molecules. Therefore, the ozone molecules generated in the discharge device 20 are flowing down into the sterilization chamber 30 for disinfections of the medical tools, manufactured drugs and biologically contaminated articles. The remaining ozone after sterilization treatment in the sterilization chamber 30 exhausts into the ozone destruction device 60 before it is discharged into open space. The main frame 10 of the apparatus is made of a non-oxidizing, thermally conductive material, such as ceramics, ferrous or non-ferrous metals, preferably stainless steel. The discharge device 20 is basically made of the corona discharge unit shown in FIG. 2, where a resistive medium 26 is inserted into the space between the grounded electrode 24 and the meshed-wire electrode 22. The typical resistive medium is natural or synthetic elastomers, such as natural rubber, silicon rubber, and urethane rubber. The grounded and meshed-wire electrodes are made of non-oxidizing and electrically conducting material, such as ferrous or non-ferrous metals, preferably stainless steel. A high-voltage electrical pulse is provided from the power input 40 to the meshed-wire electrode 22. A corona discharge 28 occurs in the space between the two electrodes, generating ozone in the oxygen gas. Although FIG. 2 shows one resistive-barrier discharge unit for simplicity, a large area and large volume discharge space is achievable by stacking layers of the resistive-barrier discharge unit, connecting each of the meshed-wire electrode in parallel and providing the high-voltage electrical power to the meshed-wire electrodes, thereby efficiently utilizing the space and generating ozone in a compact space. The grounded electrodes are also connected in parallel in the discharge device consisting of several resistive-barrier discharge units.

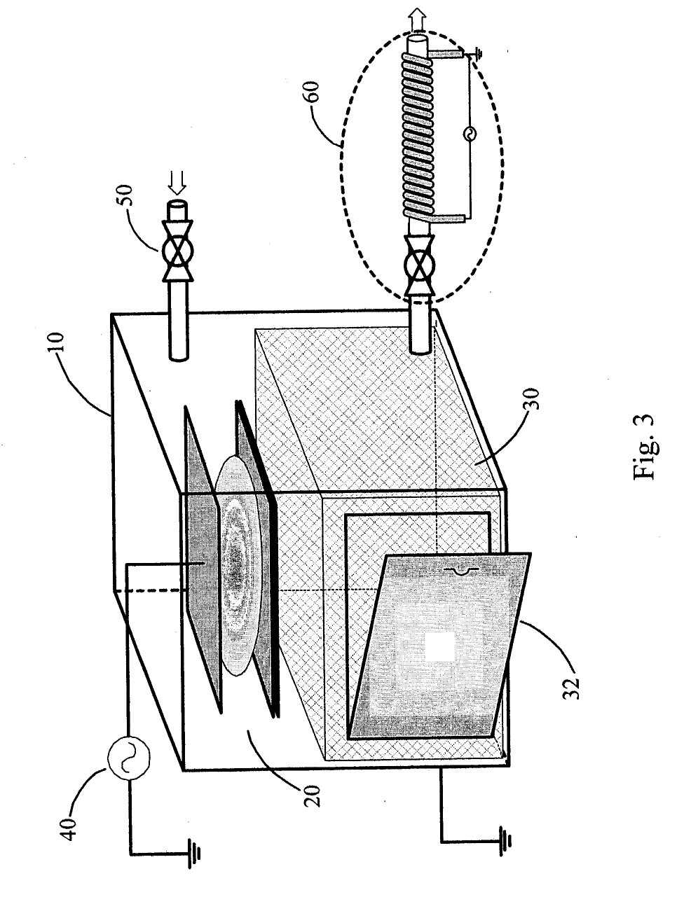

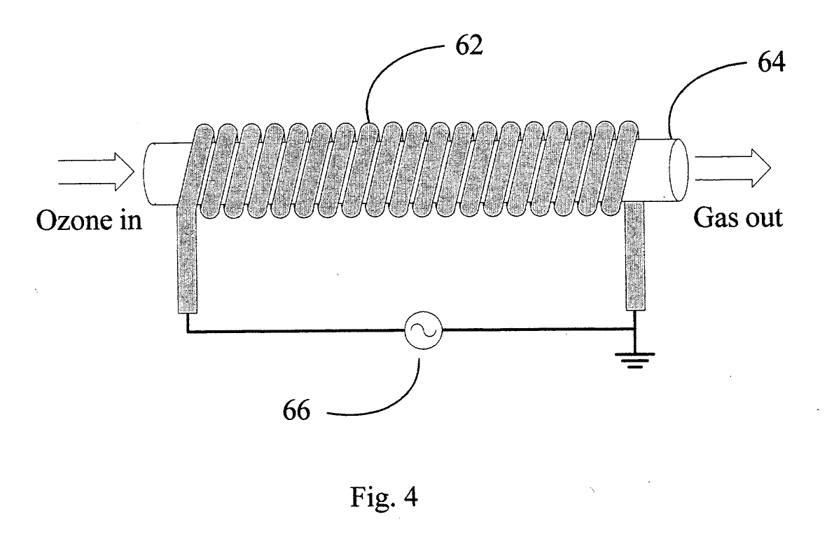

A schematic presentation of the present invention is shown in FIG. 3, where the medical tools, manufactured drugs and biologically contaminated articles are placed in the sterilization chamber 30 by opening the sterilization chamber door 32. Oxygen gas from the gas input unit 50 enters the sterilization apparatus 10 by opening the input valve in the gas input unit 50 and by opening the exhaust valve connected to the ozone destruction device 60, flushing out or bleeding other gases and maintaining only the oxygen gas inside the apparatus 10. The high-voltage electrical pulse from power input 40 provides the necessary power for electrical discharge 20. The apparatus 10 is sealed from outside during sterilization process by closing the sterilization chamber door 32, and by closing the input valve in the gas input unit 50 and the exhaust valve connected to the ozone destruction device 60. The remaining ozone after the sterilization enters the ozone destruction device 60 as shown in FIG. 4, where the heating coil 62 wraps around the heating tube 64 made of quartz or pyrex. The heating coil 62 is operated by an electrical power source 66, which can be either dc or ac operating mode for convenience. The ozone is instantly disintegrated into oxygen molecules when the temperature inside the heating tube is higher than 200 degree Celsius. Therefore, the residual ozone from the sterilization process can be effectively eliminated before discharged into open space.

Sterilization experiment was carried out for the discharge device consisted of four layers of the resistive-barrier discharge unit. The total volume of the sterilization apparatus 10 including discharge device 20 and sterilization chamber 30 was 100 liters (0.1 m³). The wall of the sterilization apparatus 10 in this experiment was made of stainless steel. The discharge area of each resistive-barrier discharge unit was 400 cm² (20 cm×20 cm). The resistive medium used in the experiment was silicon rubber sheet of approximately 2 mm thickness. The discharge space gap between the silicon rubber surface 26 and the meshed-wire electrode 22 was approximately 5 mm. The grounded and meshed-wire electrodes in this experiment were made of stainless steel. Many different types of resistive medium had been tried in the experiment. The silicon rubber sheet of approximately 2 mm thickness was the best in the sterilization performance for a 12 kV electrical source. The silicon rubber sheet used in this experiment is the silicon sheet from Youngil Silicon Co. in Korea. This silicon sheet is very stable in its physical and chemical properties in the temperature range from minus 100 degree Celsius to 250 degree Celsius, and is a non-oxidizing and good thermal-conducting material. It is acid- and alkali-resistant, is also resistant against arcing, and is non-burnable due to lack of hydrocarbon in its composition. The silicon sheet is also safe to use without any danger of toxic emission during operation.

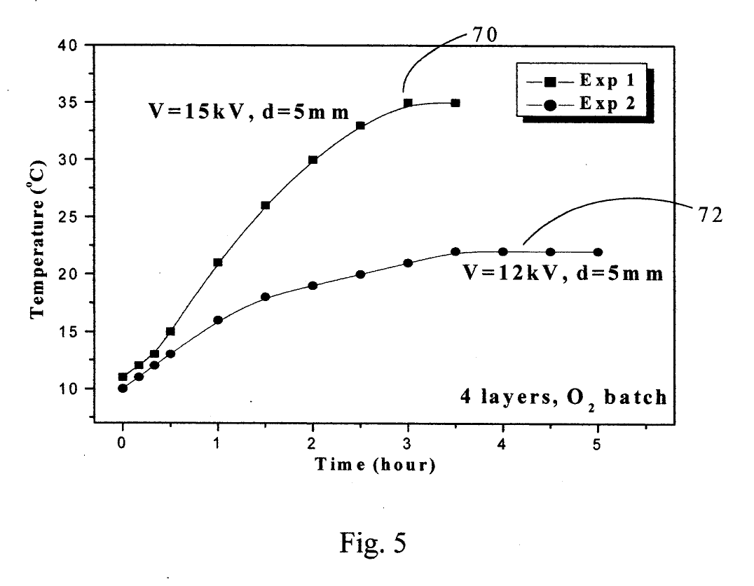

One of the most important parameters to be considered for the low temperature sterilization is the temperature in the sterilization chamber 30. Shown in FIG. 5 is the experimental measurement of the temperature versus time for the electrical voltage of 12 kV and 15 kV applied, respectively, to the meshed-wire electrode for corona discharge. Note that the ac electrical source used in the experiment was an inexpensive commercially available transformer with capacity of 15 kV, 20 mA and 60 Hz. The room temperature during the experiment for FIG. 5 was 10 degree Celsius. Gas used in the experiment was oxygen. The electrical power used for the 12 kV discharge voltage was about 60 W, whereas that for the 15 kV discharge voltage was about 95 W. The temperature in FIG. 5 increases and then saturates for both cases. The saturation temperature for 12 kV case was 23 degree Celsius as shown in the curve 72 and that for 15 kV was 35 degree Celsius as shown in the curve 70. Most of the sterilization experiments were carried out by the 12 kV discharge voltage. Obviously, the apparatus of the present invention is suitable for the low temperature sterilization of medical tools, manufactured drugs and biologically contaminated articles. The apparatus size and electrical power depend on the daily volume of sterilization material for each application. For example, the manufactured drug sterilization in a pharmaceutical company may need a large size of sterilization apparatus due to a large amount of manufactured drugs. On the other hand, the medical tool sterilization in a hospital may need a moderate size of the sterilization apparatus.

A gas cooling device can be optionally attached to the sterilization chamber 30 in case when the gas temperature inside the sterilization chamber 30 is so high that the low temperature sterilization is not possible. The gas cooling device with its gas input and output tubes attached to the sterilization chamber wall consists of a coiled gas tube made of a non-oxidizing, thermally conducting material, which is cooled by any means such as air cooling or water cooling jacket. The gas inside the sterilization chamber is circulated through this optional gas cooling device. The temperature in the sterilization chamber can be adjusted to any desired level for proper operation by this optional gas cooling device.

The gas temperature inside the sterilization chamber 30 can also be reduced by using the main frame wall 10 as a heat radiator. The stainless steel wall of the main frame 10 can be used as a grounded electrode 24 in FIG. 2, where the resistive medium 26 is overlaid on the inside surface of the stainless steel wall. The meshed-wire electrode covers the resistive barrier with 5 mm of the gap distance from the surface of the resistive medium. The heat generated from the electrical discharge is trapped in the discharge space, which is in the vicinity of the stainless steel wall. Note that steel is a good thermal conductor, emitting the heat out of the sterilization chamber. This system configuration may further reduce the gas temperature in the stainless steel chamber.

Ethylene oxide is very powerful oxidizing material. It can sterilize the medical tools within 2 hours. But, evaporation of the residual EO material on the sterilized medical tools in an aerator requires more than 15 hours. Therefore, the required time for sterilization of the medical tools by EO in a hospital is about 17 hours. In order to compare the sterilization effectiveness of the present invention with the conventional EO sterilization, the biological indicator called Verify®SCBIs was placed inside the sterilization chamber 30 of the present invention. The verification ampule was buried in the hospital cloth with depths more than 8 cm for simulation of the medical tool sterilization in a hospital. The first ampule was sterilized within 10 hours with the operation conditions of the 12 kV discharge voltage, four layers of discharge unit and the oxygen input gas. The sterilization was positively confirmed. The sterilization confirmation experiment was repeated by reducing the residence time of the ampule in the sterilization chamber 30. The ampule with 5 hours of residence time confirms the positive sterilization. However, the ampule with 4 hours of residence time failed in confirmation process of sterilization. Sterilization verification experiment was also carried out for the ampule packaged in wrapping papers for simulation of the manufactured drug sterilization. The ampule wrapped in papers with 3 hours of residence time in the sterilization chamber 30 was confirmed to be completely sterilized. This experiment confirms positively of the low temperature sterilization of the medical tools and manufactured drugs by the present invention within 5 hours of sterilization time. The sterilization experiment was carried out for the apparatus volume of 100 liters where the sterilization chamber volume is 60 liters and the discharge device volume is 40 liters. The sterilization of this experiment requires a 60 W power for 5 hours of residence time. For a given sterilization time, the required electrical power is proportional to the apparatus volume. Therefore, the power required increases to n times of 60 W if the apparatus volume increases to n times of 100 liters.

Although the experiment of the present invention has been carried out for dry oxygen gas, the best sterilization may occur in an optimum humidity. It is therefore recommended to find the optimal humidity obtained by routine experiment and to prepare the cleaned medical tools for this optimal humidity by preconditioning them with a proper level of dryness. It is also recommended to mix the gaseous antimicrobial agent or hydrogen peroxide with oxygen to improve disinfection mechanism.

The sterilization experiment of the verifying ampule was also carried out for air instead of oxygen gas. The sterilization effectiveness of the present invention for the air as a working gas is not as good as that for the oxygen gas. The sterilization time in air is typically twice of that in oxygen. The temperature inside the sterilization chamber 30 is higher than the outside temperature. Therefore, water condenses on the inside wall surface in the sterilization chamber 30 when the air is used as a working gas, which contains water molecules. The pH test of the water condensed on the wall indicates that it is a weak nitricacid solution, which may have harmful effects on human body. Therefore, sterilized tools and sterilized manufactured drugs must not be in contact with this water. In this context, it is recommended to use the oxygen gas for the present invention. Most hospitals have oxygen lines, if there are no oxygen lines, oxygen tanks are also easily available.

Although this embodiment is the apparatus for sterilization of the medical tools and manufactured drugs by making use of the resistive-barrier discharge with silicon rubber

What is claimed is:

1. An apparatus for sterilization of medical tools in hospitals or manufactured drugs in CLAIMS pharmaceutical manufacturing and packaging plants or biologically contaminated articles, said apparatus comprising:
(a) a large sealed and electrically grounded, non-oxidizing metallic box attached to a valved oxygen source at the upper section of said box and attached to a combination of a valved exhaust system and an ozone destruction device at the lower section of said box;
(b) the upper interior section of said box contains an ozone generating electrical discharge device comprising alternatively arranged layers of meshed-wire electrode being connected in parallel and being chargeable with a high-voltage, and layers of grounded electrode covered with a resistive elastomer material; and
(c) the lower interior section of said box contains a sterilization section with an excess door to place a material to be sterilized into said section, wherein germs, viruses and pathogenic bacteria are killed by the exposure to ozone mixed with oxygen gas.

2. In the apparatus according to claim 1, wherein said ozone destruction device comprising a heating coil wrapping around a heating tube.

3. In the apparatus according to claim 1, wherein said elastomeric discharge device consists of silicon rubber sheet of approximately 2 mm thickness, and spacing of approximately 5 mm between said meshed-wire electrode and said rubber sheet.

4. In the apparatus according to claim 1, wherein said meshed-wire electrode in said discharge device is provided with a 60 Hz alternating current source operating with voltage capacity of 10 kV to 30 kV and current capacity of 20 mA to 100 mA.

5. In the apparatus according to claim 1, said box wall made of stainless steel serves a grounded electrode for further reduction of the temperature in said sterilization chamber.

6. A process for sterilization of medical tools or manufactured drugs or biologically contaminated articles in a sterilization chamber, comprising:
(a) placing medical tools or manufactured drugs or biologically contaminated articles with or without packaging in a sealed ozone generating sterilization chamber;
(b) containing an ozone generating electrical discharge device comprising alternatively arranged layers of meshed-wire electrode chargeable with a high voltage and layers of grounded electrode which is covered with a resistive elastomeric material;
(c) introducing oxygen gas into said electrical discharge device of said sterilization chamber, at least partially flushing or bleeding out the remaining air in said chamber;
(d) closing all valves in order to seal said discharge device and sterilization chamber from outside;
(e) initiating the electrical discharge in said discharge device and generating ozone, which in turn kills germs, viruses, and pathogenic bacteria in said chamber;
(f) upon completion of said sterilization, turning on a heating coil of an ozone destruction device which is attached to an exhaust pipe; and
(g) exhausting the gas mixed with ozone into said ozone destruction device by opening a gas exhaust valve attached to said sterilization chamber.

7. In the process according to claim 6, wherein said resistive elastomeric device consists of silicon rubber sheet of approximately 2 mm thick and spacing of approximately 5 mm between said meshed-wire electrode and said rubber sheet.

8. In the process according to claim 6, wherein said discharge device operates at the voltage range from 12 kV to 15 kV and at the power range from 60 W to 100 W provided by a 60 Hz electrical source, and are capable of handling 60 liters of sterilization chamber capacity, which can sterilize said medical tools or manufactured drugs or biologically contaminated articles within 5 hours at a temperature less than 35 degree Celsius.

9. In the process according to claim 6, wherein said discharge device operates at the voltage range from 12 kV to 15 kV and the power range from n times of 60 W to n times of 100 W provided by a 60 Hz electrical source, and are capable of handling n times of 60 liters of sterilization chamber, which can sterilize said medical tools or manufactured drugs or biologically contaminated articles within 5 hours at the temperature less than 35 degree Celsius, wherein n represents a desired multiple of basic sterilization capacity of 60 liters.

10. In the process according to claim 6, wherein said medical tools wrapped in hospital cloths or manufactured drugs in typical packaging materials or biologically contaminated articles are kept at the optimum humidity for best sterilization by preconditioning their dryness before storing them in said sterilization chamber.

11. In the process according to claim 6, wherein the oxygen gas is mixed with a gaseous antimicrobial agent or mixed with hydrogen peroxide for improvement of disinfection mechanism.
